(19) United States
(12) Patent Application Publication
(10) Pub. No.: US 2009/0233885 A1
(43) Pub. Date: Sep. 17, 2009

(54) Compositions and Methods for Fat Reduction

(76) Inventor: Diane I. Duncan, Fort Collins, CO (US)

Correspondence Address: CR MILES, P.C.;CRAIG R. MILES, 405 MASON COURT, SUITE 119, FORT COLLINS, CO, 80524, US

(21) Appl. No.: 11/885,084

(22) PCT Filed: Jul. 13, 2007
(86) PCT No.: PCT/US2007/015946

§ 371 Date: May 18, 2009

Related U.S. Application Data
(60) Provisional application No. 60/830,947, filed on Jul. 14, 2006.
(60) Provisional application No. 60/860,838, filed on Nov. 22, 2006.

(51) Int. Cl. A61K 31/661

(52) U.S. Cl. 514/121

(57) ABSTRACT

Compositions, methods, and apparatuses for treatment of subcutaneous fat tissue for the purpose of fat tissue reduction or other alterations of the subcutaneous fat tissue which affect the appearance of the overlying skin layer.

This application is the United States National Stage Application of International Patent Cooperation Treaty Application No. PCT/U.S.07/015,946, filed Jul. 13, 2007, which claims the benefit of U.S. Provisional Patent Application No. 60/830,947, filed Jul. 14, 2006, and which claims the benefit of U.S. Provisional Patent Application No. 60/860,838, filed Nov. 22, 2006, each hereby incorporated by reference herein.

### I. TECHNICAL FIELD

The invention relates to compositions, methods, and apparatuses for treatment of subcutaneous fat tissue for the purpose of fat tissue reduction or other alterations of the subcutaneous fat tissue which affect the appearance of the overlying skin layer.

### II. BACKGROUND

In 1959, phosphatidylcholine (hereinafter “PC”) was isolated and used intravenously in Odessa, Russia, for the treatment of fat embolism. In 1988, Sergio Maggiore reported use of PC injections for cosmetic purposes. PC has also been used in treating xanthelasmas in Europe and in South America. In 1995, Dr. Patricia Rittes is believed to be the first to use subcutaneous injections of PC for the purpose of fat reduction. An injectable form of PC (LIPOSTABIL®, Sanofi-Aventis, Brigewater, N.J.) has been indicated for treatment of fat embolisms, coronary artery plaque, and fat tissue.

PC is often an ingredient in injectable fat reducing formulas. When isolated, it is produced as a powder. When reconstituted, it is quite viscous and must be mixed with a detergent, such as sodium deoxycholate (hereinafter “DC”), to solubilize it sufficiently to create an injectable form. DC is a bile salt that can function to make the PC soluble in water or other biocompatible solvents; otherwise, the PC can precipitate out of solution. DC has been described as having a “detergent” effect on fat dissolution in a porcine in vitro study and has nonspecific effects on both adipose and muscle cells. Other pharmaceuticals, such as Fungizone (Bristol Myers Squibb, New York, N.Y.) (an injectable form of amphotericin B), are commonly combined with bile salts to enhance their solubility and make them compatible with intravenous delivery.

While conventional formulations of DC without PC (“DC formulations”) and conventional formulations of PC combined with DC (“PC/DC formulations”) have been shown to achieve a level of fat reduction, substantial unresolved problems remain with the use of such conventional formulations and conventional methods of use.

A first significant problem with conventional DC formulations may be a marked prolonged inflammatory reaction along with excess fibrosis and collagen formation post injection. A strong histamine release may result within five to ten minutes post injection of DC formulations. Onset of swelling may be observed within thirty minutes of injection and appears to be a dose related reaction. Burning and pain following injection of DC formulations may persist for a month or more. Referring primarily to FIG. 1, in vivo histopathological studies of tissue segments injected with 4.2% DC indicate moderate inflammation of the overlying dermis and inflammation of the eccrine sweat glands one month post injection (the pointer directed to a region exhibiting the general condition). Marked vasculitis may also occur as shown in FIG. 2 (the pointer directed to a region of the vessel exhibiting the general condition). Conventional DC formulations when diluted may reduce the side effects of pain and burning; however, the efficacy of fat reduction may also be substantially reduced.

A second significant problem with the use of DC formulations may be the failure to disperse sufficiently through fat tissue to avoid localized cavitation about the sites of injection or avoid areas of untreated uncavitated fat tissue between the injection sites. The failure of such conventional DC formulations or conventional PC/DC formulations to disperse between injection sites can result in an uneven layer of fat tissue supporting the skin layer which can feel or have an appearance of unevenness or lack of uniformity.

As shown in FIG. 3, a 4.2% DC formulation including 0.2 cubic centimeter (“cc”) methylene blue per 10 cc of the DC formulation administered 0.5 cc per injection site at a depth of 10 millimeters (“mm”) with injection sites 1.5 centimeters (“cm”) apart resulted in a pattern of localized dispersion of the DC proximate to the injection sites (dark colored areas-indicated by pointers) interrupted by areas in which the DC formulation has not dispersed (light colored areas) in tissue specimens harvested between about 15 minutes and even after one hour post injection.

One approach to generating a greater level of dispersion has been to utilize formulations of PC 5.0%-DC 4.2%. However, PC can cause cholinergic side effects such as nausea, vomiting, diarrhea, flushing, sweating, bradycardia, and the like when the total administered amount of PC exceeds about 2000 mg per treatment. The use of PC 5.0%-DC 4.2% in the context of the dose restriction may severely limit the size of the tissue region treated in a single session. To overcome this problem, practitioners may dilute the formula for example to PC 2.5%-DC 2.1%. However, diluted formulas utilized as above-described can result in a similar pattern of localized dispersion (dark colored areas-indicated by pointers) of the PC 2.5%-DC 2.1% proximate to the injection sites as shown by FIG. 4.

A third significant problem with the use of conventional PC/DC formulations may be that the characteristics or level of lipolytic action achievable may not be optimal. As above-described, certain conventional formulations may require multiple sessions because the concentration of PC in the formulation may limit the size of the tissue region treated in a single treatment. Alternately, certain conventional formulations when diluted to overcome this problem may also exhibit the above-described problem of having a localized dispersion pattern about the injection locations and can also exhibit a reduced level of lipolysis.

A fourth significant problem with the use of conventional PC/DC formulations can be that conventional ratios or concentrations of PC to DC may not be optimal. Prior to the discovery of the inventive formulations described herein, it is believed that it was not known and that the conventional teachings regarding DC formulations and PC/DC formulations did not disclose (whether expressly or inherently) the strong effect which the ratio of PC to DC in a formulation can have on the level of dispersion of the PC/DC formulation in subcutaneous fat tissue or the level of lipolytic action of the PC/DC formulations on subcutaneous fat tissue, or did not teach the ratios of particular PC/DC compositions described herein (or certain concentrations of PC/DC in a biocompatible solvent which provide such PC to DC ratios), or did not teach that the ratios or concentrations of the PC/DC compositions described herein had a greater efficacy with respect to dispersion or lipolysis in subcutaneous fat tissue within a broader range of PC/DC formulations.

The inventive PC/DC compositions and methods of using such PC/DC compositions described herein address each of the long felt but unresolved problems with the use of conventional DC formulations and conventional PC/DC formulations for the treatment of subcutaneous fat tissue, and provide PC/DC compositions having PC to DC ratios which exhibit a increased level of dispersion and lipolytic action in subcutaneous fat tissue across a range of PC and DC concentrations.

### III. DISCLOSURE OF INVENTION

Accordingly, a broad object of the invention can be to provide PC/DC compositions providing certain ratios of PC to DC weight to weight (wt/wt), or certain ratios of PC to DC wt/wt at certain concentrations, in a biocompatible solvent which can be administered by injection into subcutaneous fat tissue, and which can have one or more advantages of increased lipolytic activity, reduced inflammatory response, or increased dispersion characteristics, whether separately, collectively or in various permutations and combinations, as compared to conventional DC formulations or conventional PC/DC formulations.

Another broad object of the invention can be to provide compositions for injection having specific ratios of PC to DC for fat reduction which can provide increased lipolytic activity, a reduced inflammatory response, or increased dispersion characteristics which can further include isoproterenol hydrochloride (“ISUPREL™”); collagenase, such as Clostridial collagenase; or an amount of one or more of: nicotinic acid, clofibrate, tannic acid, scorpion toxin, snake venom, beta adrenergic stimulants, dimethlyaminoethanol, hyaluronic acid, penta-O-galloyl-alpha-D-glucose, hormone sensitive lipase, human adipose triglyceride lipase, tnf-alpha, raspberry ketone, ethanol, rosiglitazone, peroxisome-proliferator activated receptor gamma, Y-9738 (ethyl 2(4-chlorophenyl)-5-ethoxy-4-oxazoleacetate) oliphen, fish oil, scallop shell extract, peanut shell extract, and caffeine, separately or in various permutations and combinations.

Another broad object of the invention can be to provide methods of utilizing the inventive PC/DC compositions including without limitation utilizing an injection location identification template which can engage the surface of the skin overlaying the subcutaneous tissue to be injectably treated to identify the location of the plurality of injection locations on the surface of the skin (also referred to as a “skin layer surface”).

Naturally, further objects of the invention are disclosed throughout other areas of the specification, drawings, photographs, and claims.

### IV. BRIEF DESCRIPTION OF THE DRAWINGS

FIG. 1 is an image of the dermis overlying a DC only injection site one month post injection showing moderate inflammation, inflammation of the eccrine sweat glands and vasculitis.

FIG. 2 is an image of the subcutaneous fat tissue one month post injection of DC only showing loss of vessel viability and intimal edema (lower left corner of photograph).

FIG. 3 is an image of a pattern of localized dispersion of a conventional DC formulation proximate to injection sites (dark colored areas) interrupted by areas in which the DC formulation has not dispersed (light colored areas) in tissue specimens harvested between 15 minutes and one hour post injection of 0.5 milliliter (“mL”) of 4.2% DC in saline at each of a plurality of injection sites about 1.5 cm apart.

FIG. 4 is an image of a pattern of localized dispersion of a conventional PC/DC formulation proximate to injection sites (dark colored areas) interrupted by areas in which the PC/DC formulation has not dispersed (light colored areas) in tissue specimens harvested between 15 minutes and one hour post injection of 0.5 milliliter (“mL”) of PC 2.5% /DC 2.1% in saline at each of a plurality of injection locations about 1.5 cm apart.

FIG. 5 is an image of a pattern of substantially uninterrupted dispersion of a particular embodiment of an inventive PC/DC composition in tissue specimens harvested between 15 minutes and one hour post injection of 0.5 mL of a particular embodiment of the inventive PC/DC compositions (PC 2.66% (26.6 mg/mL)/DC 2.48% (24.8 mg/mL) in saline) at each of a plurality of injection locations about 1.5 cm apart.

FIG. 6 is a plan view of an embodiment of an injection location identification template.

FIG. 7 is a side view of an embodiment of an injection location identification template.

FIG. 8 is an end view of an embodiment of an injection location identification template.

FIG. 9 is a drawing which shows a person exhibiting marks viewable upon disengaging the injection location identification template from the surface of the skin each mark identifying a corresponding h injection location at which an amount of a PC/DC composition can be injectably delivered to the subcutaneous fat layer.

FIG. 10 is a schematic drawing which shows particular embodiments of an inventive method of injecting PC/DC composition into subcutaneous fat tissue for lipolysis.

FIG. 11 is a schematic drawing which shows a particular embodiment of an inventive method of injecting PC/DC compositions into subcutaneous tissue for treatment of cellulite.

FIG. 12 is a schematic drawing which shows a particular embodiment of an inventive method of injecting PC/DC compositions into subcutaneous tissue to affect skin retraction.

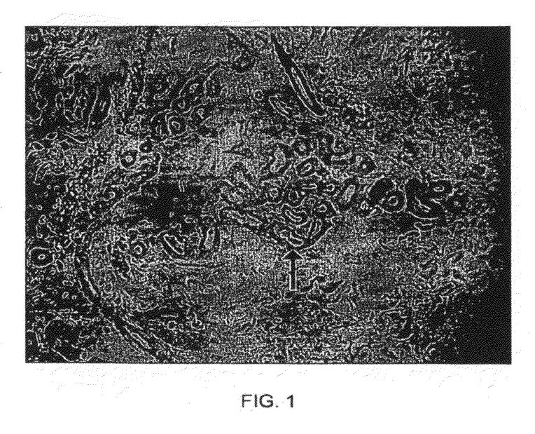

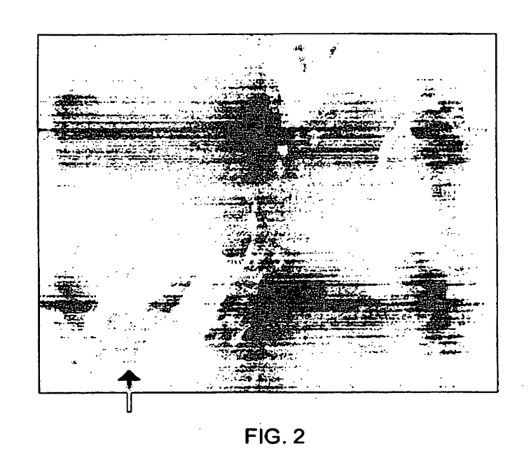

### V. MODE(S) FOR CARRYING OUT THE INVENTION

Generally, injectable compositions effective in reducing subcutaneous fat which provide an amount of phosphatidylcholine and an amount of deoxycholate in a ratio of between about 1.0:0.88 (wt/wt) and about 1.0:1.1 (wt/wt) in an amount of biocompatible solvent. Specifically, injectable compositions effective in reducing fat which provide an amount of phosphatidylcholine and an amount of deoxycholate in a ratio of between about 1.0:0.90 (wt/wt) and about 1.0:0.95 (wt/wt) in an amount of biocompatible solvent.

Various efficacious embodiments of a PC/DC composition can be prepared by combining an amount of PC with an amount of DC in ratios between about 1.0:0.88 and about 1.0:1.0 (wt/wt) and dissolving the combination of the amount of PC and the amount of DC in a biocompatible solvent. The biocompatible solvent selected can be any material, without limitation and by way of example, any one of: an amount of water, an amount of saline (typically an isotonic solution of sodium chloride and distilled water), an amount of water combined with an amount of alcohol (typically added as a preservative), an amount of saline combined with an amount of alcohol, an amount of water combined with an amount of benzyl alcohol, an amount of saline combined with an amount of benzyl alcohol, or the like.

Certain embodiments of the inventive PC/DC compositions can provide specific PC to DC ratios within the broader range of ratios above-described and can include PC/DC compositions having a ratio of PC to DC of: between about 1.0:0.88 to about 1.0:0.89, between about 1.0:0.89 to about 1.0:0.90, between about 1.0:0.90 to about 1.0:0.91, between about 1.0:0.91 to about 1.0:0.92, between about 1.0:0.92 to about 1.0:0.93, between about 1.0:0.93 to about 1.0:0.94, between about 1.0:0.94 to about 1.0:0.95, between about 1.0:0.95 to about 1.0:0.96, between about 1.0:0.96 to about 1.0:0.97, between about 1.0:0.97 to about 1.0:0.98, between about 1.0:0.98 and about 1.0:0.99, between about 1.0:0.99 and about 1.0:1.0, and between about 1.0:1.1. These ratios may also be expressed as the quotient of the amount of DC divided by the amount of PC. For example, 24.8 mg DC divided by 26.6 mg PC results in a quotient of about 0.93 and the ratio for this embodiment of the inventive PC/DC invention can be expressed in the alternative as either: 1.0:0.93 or 0.93.

Embodiments of the inventive PC/DC compositions having the ratios above-described can provide an amount of PC of between about 24 milligrams per milliliter (“mg/mL”) and about 52 mg/mL of the biocompatible solvent, or between about 24 mg/mL and about 28 mg/mL of the biocompatible solvent, or between about 26 mg/mL and about 27 mg/mL of the biocompatible solvent can be provided depending upon the application.

Correspondingly, these embodiments of the PC/DC compositions having the ratios above-described can provide an amount of DC adjusted to generate the desired ratio of between about 22 mg/mL and about 42 mg/mL of the biocompatible solvent, or between 22 mg/mL and about 26 mg/mL of the biocompatible solvent, or between about 24 mg/mL and about 25 mg/mL of the biocompatible solvent can be provided depending upon the application. Additionally, certain embodiments of the inventive PC/DC compositions can have specific ratios such as 0.9323 (PC 26.6 mg/mL-DC 24.8 mg/mL) or 0.9688 (PC 25.6 mg/mL-DC 24.8 mg/mL) in the biocompatible solvent.

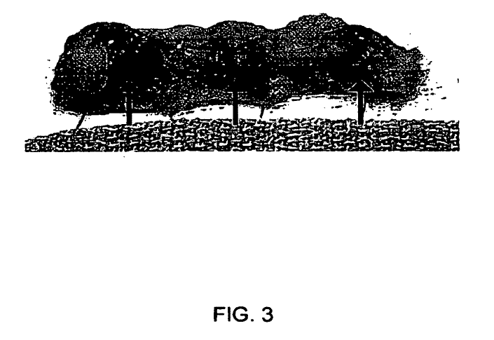

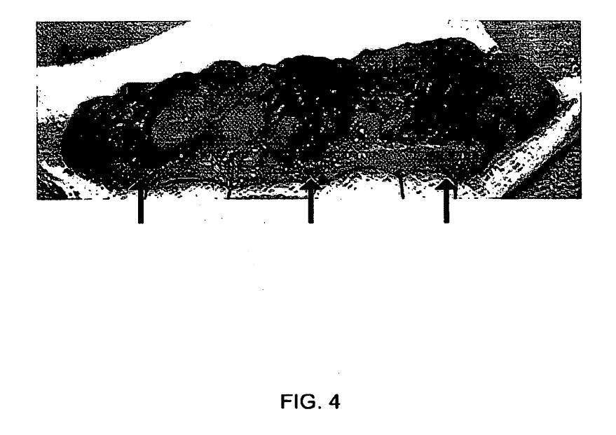

As to each of the above-described PC/DC compositions and other PC/DC compositions which are encompassed by the inventive PC/DC ratios above-described, the inventive PC/DC ratios are each at or slightly below 1 and are not less than about 0.88. These inventive PC/DC ratios are based upon the discovery that a PC/DC composition having a PC/DC ratio below about 0.88 may not yield or yields a reduced lipolytic activity as compared to PC/DC compositions which provide a PC/DC ratio at or greater than 0.88, and that a PC/DC composition have a PC/DC ratio of greater than 1.0 can yield a composition characterized by localized dispersion in the subcutaneous fat layer about the injection location, or a reduced dispersion in the subcutaneous fat layer, or a dispersion that results in substantial “skip areas” between injection sites as shown in FIGS. 3 and 4 and as above-described, or produces a greater inflammation of the overlying dermis, inflammation of the eccrine sweat glands or vasculistis as compared with the PC/DC compositions having the inventive PC/DC ratios.

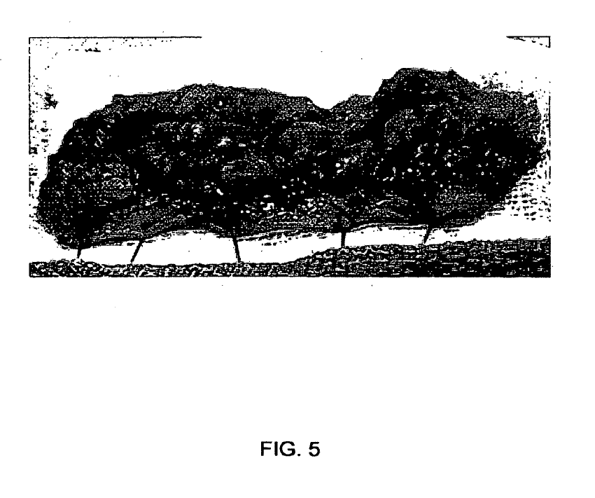

Now referring primarily to FIG. 5, a particular embodiment of the inventive PC/DC compositions having a PC/DC ratio of about 0.93 which provides about 2.6% PC and about 2.5% DC (as to the particular embodiment of the PC/DC composition utilized in the example PC 26.6 mg/mL and DC 24.8 mg/mL of saline) administered 0.5 cc per injection site at a depth of 10 mm with injection sites 1.5 cm apart can result in a pattern of dispersion uninterrupted between injection sites in tissue specimens harvested between about 15 minutes and about one hour post injection.

Any particular embodiment of the PC/DC compositions can further include an amount of anesthetic. Various anesthetics individually or in combination may be included in a particular embodiment of a PC/DC composition based on the application such as: ropivacaine, articaine, benzocaine bupivacaine, chloroprocaine, etidocaine, hexylcaine, lontocaine, lidocatine, levobuivaciaine, mepivacaine, prilocaine, procaine, and tetracaine, it is not intended that the invention be limited by including an anesthetic, or limited to including one or more of the anesthetics described herein. Rather, the anesthetics described herein are intended to provide examples of the numerous and varied anesthetics which may be included in inventive embodiments of the PC/DC compositions depending upon the application. As to particular embodiments of the PC/DC compositions an amount of ropivacaine which provides between about 4 cc and about 6 cc in 100 cc of the PC/DC composition can be utilized. For example, an amount of ropivacaine which provides 5 cc can be utilized per 100 cc of a PC/DC composition.

Similarly, any particular embodiment of the PC/DC compositions can further include an amount of beta adrenergic stimulator. A beta adrenergic stimulator can bind either directly or indirectly to the beta-receptor, thereby stimulating it. The stimulated receptor triggers a complex series of events involving multiple enzyme systems which results in an accumulation of cyclic AMP within the cell and decreased ATP. These conditions can activate lipases which break down triglyceride fats in the adipocytes into free fatty acids, which can be used by the cell for growth and metabolism, or may be discharged extracellularly. While various beta adrenergic stimulators individually or in combination can be included in particular embodiment of the PC/DC compositions such as isoproterenol hydrochloride (ISUPREL™), isoproterenol hydrochloride, forskolin, norepinephrine, guarana and clenbuterol, or other beta-receptor specific agonist (or non-specific agonists such as ephedrine as to certain applications) it is not intended that the invention be limited by including any beta adrenergic stimulator, or limited to including a beta adrenergic stimulator described herein. Rather, the beta adrenergic stimulators described herein are intended to provide examples of the numerous and varied beta adrenergic stimulators which can be included in inventive embodiments of the PC/DC compositions. As to particular embodiments of the PC/DC compositions an amount of isoproterenol hydrochloride which provides between about 4 mg and about 6 mg in 100 cc of the PC/DC composition can be utilized. For example, an amount of isoproterenol hydrochloride which provides 5 mg can be utilized per 100 cc of the PC/DC composition.

Again, any particular embodiment of the PC/DC compositions can further include, individually or in various permutations or combinations, an amount of collagenase, such as Clostridial collagenase or an amount of one or more of nicotinic acid, clofibrate, tannic acid, scorpion toxin, snake venom, beta adrenic stimulants, dimethlyaminoethanol, hyaluronic acid, penta-O-galloyl-alpha-D-glucose, hormone sensitive lipase, human adipose triglyceride lipase, tnf-alpha, raspberry ketone, ethanol, rosiglitazone, peroxisome-proliferator activated receptor gamma, Y-9738 (ethyl 2(4-chlorophenyl)-5-ethoxy-4-oxazoleacetate) oliphen, fish oil, scallop shell extract, peanut shell extract, and caffeine.

Any of the above-described PC/DC compositions can be provided as part of a kit which includes an amount of PC and an amount of DC in combination, or separately in a manner which allows combination of the amount of PC with the amount of DC, to generate the inventive PC to DC ratios above-described in an amount of biocompatible solvent which can also be provided in the kit or provided separately. Alternately, the kit can provide the amount of PC and the amount of DC already dissolved in the proper amount of biocompatible solvent to generate the inventive PC to DC ratios and concentrations of PC/DC above-described.

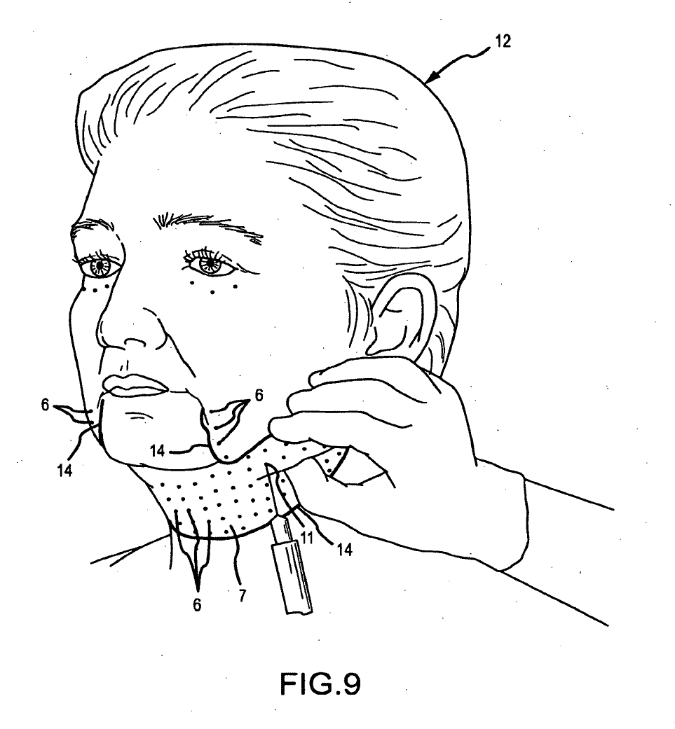

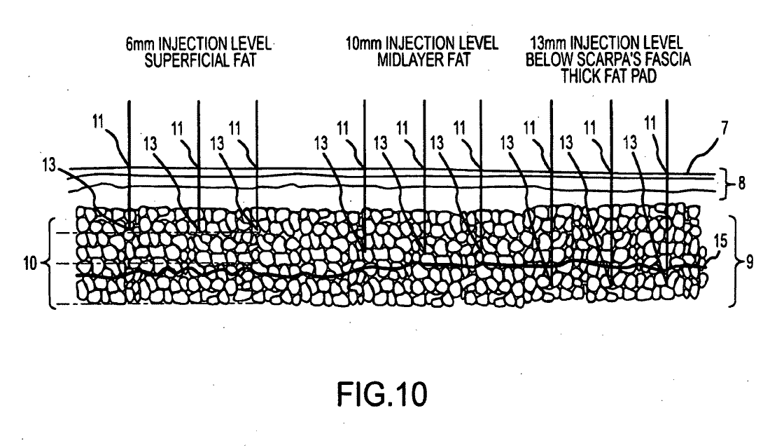

Now referring primarily to FIGS. 9-12, the inventive PC/DC compositions can be administered by locating at least one injection location (11) on a skin layer surface (7) (such as a location on the surface of the skin of a person (12)) at which the injection needle can be inserted through the skin layer (8) to establish an amount of the PC/DC composition at a level in the underlying fat tissue (9) (also referred to herein as a “fat layer”)(see FIG. 10). The term “underlying” encompasses that part of a fat tissue or a fat layer (9) located beneath the skin layer (8) injectable from a particular an injection location (11) regardless of the type of injection needle or the method of injection utilized. The term “a level” means the depth at which the PC/DC composition is established in the fat layer (9). For example, a 6 milliliter (“mm”) level means that the injection needle tip (13) has a position in the fat layer which can establish the PC/DC composition at a depth of about 6 mm into the fat layer. Naturally, in practice a plurality of injections of a PC/DC composition at a corresponding plurality of injection locations (11) intended to establish the PC/DC composition at one uniform level (10) may include an amount of variability in the actual level at which the injected amount of PC/DC composition is established depending on the injector, the type of injection needle utilized, and so forth. A reasonable variation in the level (10) between a plurality of injection locations (11) or persons (12) treated is to be understood as encompassed in the definition of any particular level (10) in the fat layer (9).

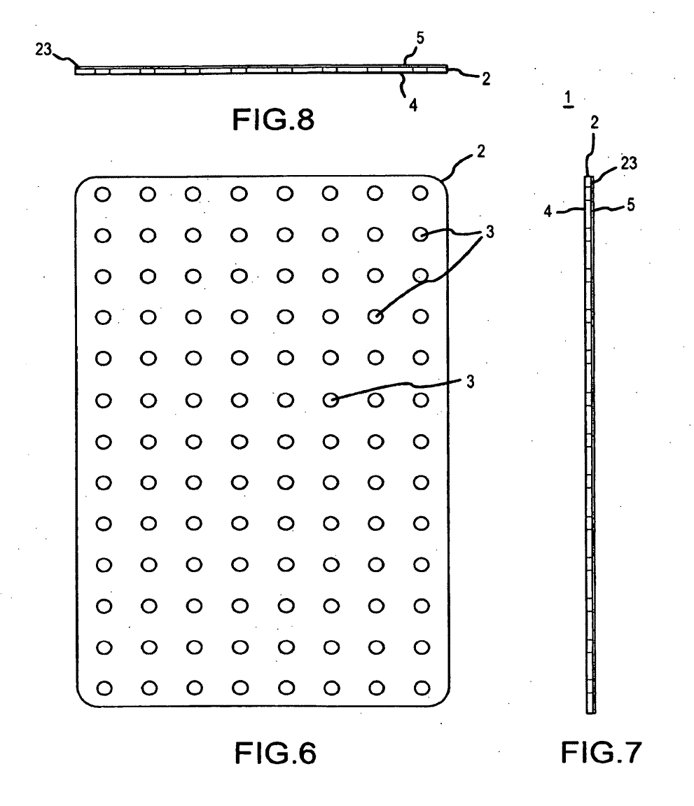

Now referring primarily to FIGS. 6-10, while a plurality of injection locations (11) can be established and the PC/DC compositions injected into the fat layer (9) underlying the skin layer (8) without the aid of any additional apparatus, a particular method of administering the PC/DC compositions above-described can in part include the use of an injection location identification template (1) (see FIG. 6). The injection location identification template (1) provides a sheet material (2) having flexure sufficient to allow engagement with a skin layer surface (7) of the skin layer (8) overlying the fat layer (9) (see FIG. 10) to be injectably treated with an embodiment of the PC/DC compositions. The injection location identification template (1) can have a plurality of apertures (3) which communicate between the surface of a first side (4) of the sheet material (2) and the surface of an opposed second side (5) of the sheet material. Each of the plurality of apertures (3) can be configured (for example in the non-limiting circular configuration shown in FIG. 6) to allow a marker (not shown) to pass through the sheet material (2) engaged with a part of the skin layer surface (7) to generate a corresponding plurality of marks (6) (see FIG. 9) on the part of the skin layer surface (7) of the skin layer (8) overlying the fat tissue or fat layer (9) to be injectably treated with any of the embodiments of the PC/DC compositions. Each of the plurality of marks (6) generated on the part of the skin layer surface (7) utilizing the injection location identification template (1) are viewable upon disengaging the injection location identification template (1) from the skin layer surface (7). Each of the plurality of marks (6) identifies an injection location (11) at which the PC/DC composition (also referred to as the “injectable composition”) can be injected by use of an injection needle or other injector to establish an amount of the PC/DC composition at one or more of a plurality of levels (10) in the fat layer (9) beneath the skin layer (8).

Numerous and varied materials can be utilized as the sheet material from which embodiments of the injection location identification template (1) so long as the sheet material has flexibility to conform sufficiently to the part of the skin layer surface (7) to allow a corresponding plurality of marks (6) to be established on the skin layer surface (7). For example the sheet material can without limitation be a plastic sheet material, a cloth sheet material, a web material, or the like in which the plurality of apertures (3) can be established. The sheet material (2) can further include an adhesive layer (23) which allows releasably fixed engagement of the sheet material (2) with the skin layer surface (7). Each of the plurality of apertures (3) of a particular injection location identification template (1) can be established a uniform distance apart of between about 0.7 cm and 1.5 cm to generate a rectilinear pattern as shown for example in FIG. 6. The injection location identification template (1) can have any manner of perimeter configuration and it is not intended that the perimeter configuration shown in FIG. 6 be limiting with respect to manner in which the injection location template can be configured.

Now referring primarily to FIG. 9-10, a person (12) may make preliminary markings (14) around the areas they want treated with the PC/DC compositions. The injection location identification template (1) may be used to generate the plurality of marks (6) on the skin layer surface (7) each of which indicates one of the plurality of injection locations (11) as shown in FIG. 9. For example, a injection location template (1) having a plurality of apertures (3) spaced uniformly about 1.5 cm apart can utilized in those instances in which the fat layer (9) to be treated underlies a relatively large area of the skin layer (8). An injection location identification template (1) having a plurality of apertures (3) spaced uniformly 1.0 cm apart can be used to treat fat layers (9) having a smaller area of comparably dense fat tissue. When treating cellulite or other skin deformities, an injection location identification template (1) having a plurality of apertures (3) spaced 0.7 cm apart can be utilized.

Now referring primarily to FIG. 10, the level (10) at which the PC/DC composition is established in the fat layer (9) can vary according to the application of the PC/DC compositions. With respect to PC/DC compositions used for lipolysis (fat reduction), if the fat layer (9) is thick, a 13-mm 20 to 32 gauge needle may be used to inject about 0.4 mL of the PC/DC composition per each of the plurality of injection locations (11) spaced about 1.5 cm apart to establish the PC/DC composition at a level (10) of about 13 mm which can be below the scarpa's facia (15). Typically, for the purpose of lipolysis a midlevel subcutaneous 0.4 mL injection of the PC/DC composition at a level (10) of about 10 mm into the fat layer (9) can be utilized. While the examples above-described and shown in FIG. 10 set out particular levels (10) in the fat layer (9), injection volume, and spacing of the plurality of injection locations (11), this is not intended to be limiting with respect to establishing the PC/DC composition at other levels in the fat layer (9), using alternate PC/DC composition injection volumes, or using alternate spacing for the plurality of injection locations (11). Rather, the examples are intended to be illustrative of a wide range of levels which at which the PC/DC compositions can be established in the fat layer (9) depending upon the particular application. In general, establishing the PC/DC composition at a level (10) in the middle of the fat layer (9) can confer an advantage as to fat reduction, while establishing the PC/DC at a level in the upper third of the fat layer (9) can confer an advantage as smoothing the skin layer (8). Depending on the application, one or more than one level (10) may be selected for the same region treated with the PC/DC compositions. Similarly, uniform spacing as opposed to non-uniform spacing of the plurality of injection locations (11) can confer an advantage in using the PC/DC compositions.

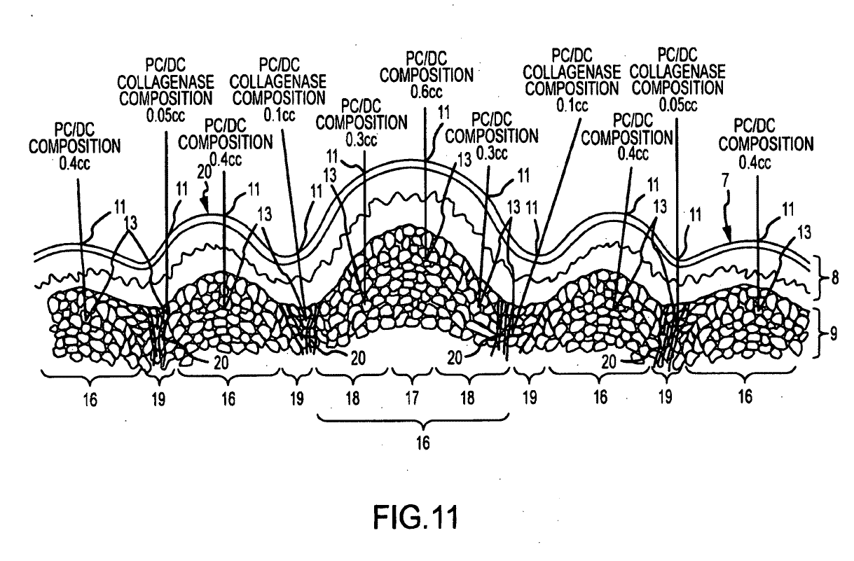

Now referring primarily to FIG. 11, an inventive method of cellulite treatment can include a treatment of the thicker areas of fat tissue (sometimes referred to as “hills” or “lumps”) (16) with the PC/DC compositions. The “hills” (16) to be treated can be identified by marking the skin surface (typically circles about the hill or lump) with addition circling of areas of the hill that are protuberant. The circles within the circles denote a thicker area of fat tissue (17) which can be treated with greater injection volume of the PC/DC composition such as about 0.6 cc per injection location (11), and the peripheral circles where thickness of the fat tissue tapers down (18) can be injected with a decreased injection volume of the PC/DC composition such as about 0.1 cc to about 0.3 cc per injection location (11). An injection location identification template (1) (or other grid or injection location identification technique) can be used to locate a plurality of injection locations (11) about 1.2 cm apart on the surface of the hill (16). Injection of the PC/DC composition at each injection location (11) at a depth of about 10 mm can be accomplished with an injection needle or mesogun. In certain embodiments of the inventive method of cellulite treatment, only the “hills” (16) are treated during the first session.

Again referring primarily to FIG. 11, an inventive method of cellulite treatment can include a treatment of the thinner areas or depressions (19) in the fat layer (9) (sometime referred to as “divots” or “valleys”). The divots (19) can be identified by marking the skin layer surface (7). The depth of the divot (19) can be correlated with the dose of a PC/DC-collagenase composition. Specifically with respect to embodiments of the PC/DC composition useful in treating divots (19), collagenase derived from Clostridium endotoxin can be further included by combining equal parts of a collagenase solution of about 250 units/mL and a PC/DC composition (the “PC/DC-collagenase composition”). A certain non-limiting embodiment of the PC/DC-collagenase composition provides a concentration of 4.5% PC and 4.2% DC in the biocompatible solvent.

The PPC-collagenase mixture can be injected at a volume of between about 0.05 cc to about 0.5 cc into the fat layer (9) underlying the divot (9) in the skin layer (8). Divots (19) of greater depth can be injected with a volume of about 0.3 cc to about 0.5 cc of the PC/DC-collagenase composition while divots (19) of lesser depth can be injected with a volume of about 0.05 cc to about 0.25 cc of the PC/DC-collagenase composition. The PC/DC-collagenase composition injection sites (11) can be about 0.5 cm apart. Injection of the PC/DC-collagenase composition can be performed with a 26 gauge ⅜″ needle which can be held at about a 45 degree angle to the divot (19) in order to better address the fibrous tissue (20). The PC/DC-collagenase composition injection location (11) can be established at the base of the divot (17), or if the divot comprises a broader depression, the injection locations (11) can be established about 0.5 cm apart.

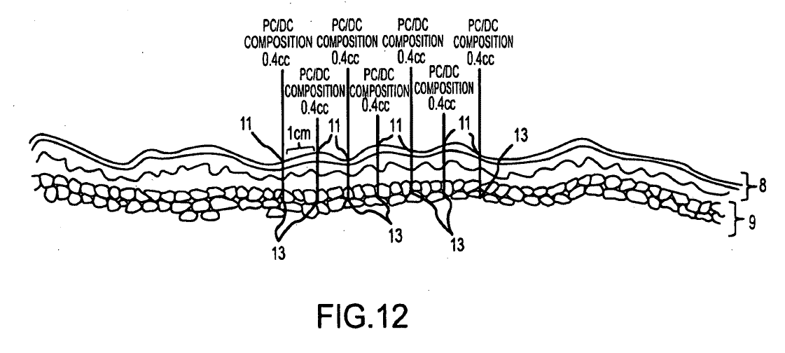

Now referring primarily to FIG. 12, if skin retraction or smoothing of the skin layer (8) is the primary goal of injecting the PC/DC compositions, a 6-mm needle may be used to inject about 0.4 mL of the PC/DC composition per each of the plurality of injection locations (11) spaced about a 1-cm apart to establish the PC/DC composition at a level of about 6 mm into the fat layer (9).

As can be easily understood from the foregoing, the basic concepts of the present invention may be embodied in a variety of ways. The invention involves numerous and varied embodiments of PC/DC compositions and methods of using such embodiments of the PC/DC compositions for the reduction of fat. This International Cooperation Treaty Application claims the benefit of U.S. Provisional Patent Application No. 60/830,947, filed Jul. 14, 2006, and U.S. Provisional Patent Application No. 60/860,838, filed Nov. 22, 2006, the entirety of each application including the description along with any photographs, figures, or tables is hereby incorporated by reference herein.

As such, the particular embodiments or elements of the invention disclosed by the description or shown in the figures or tables accompanying this application are not intended to be limiting, but rather exemplary of the numerous and varied embodiments generically encompassed by the invention or any particular part or element of the invention or equivalents thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of “injectable compositions” should be understood to encompass disclosure of the act of “injecting compositions”—whether explicitly discussed or not—and, conversely, were there effectively disclosure of the act of “injecting compositions”, such a disclosure should be understood to encompass disclosure of “injectable compositions” and even a “means for injecting compositions.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) each of the PC/DC compositions disclosed and described herein, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally any claims set forth in this specification are intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. An injectable composition for fat reduction, comprising:
a) an amount of a biocompatible solvent;
a) an amount of phosphatidylcholine; and
b) an amount of deoxycholate, wherein said amount of phosphatidylcholine and said amount of deoxycholate have a ratio of between about 1.0:0.88 and about 1.0:1.1 (wt/wt) in said amount of biocompatible solvent.

2. The injectable composition for fat reduction of claim 1, wherein said ratio of said amount of phosphatidylcholine and said amount of deoxycholate is selected from the group consisting of: between about 0.88 to about 0.89, between about 0.89 to about 0.90, between about 0.90 to about 0.91, between about 0.91 to about 0.92, between about 0.92 to about 0.93, between about 0.93 to about 0.94, between about 0.94 to about 0.95, between about 0.95 to about 0.96, between about 0.96 to about 0.97, between about 0.97 to about 0.98, between about 0.98 and about 0.99, and between about 0.99 and about 1.0.

3. The injectable composition for fat reduction of claim 2, wherein said amount of phosphatidycholine provides between about 24 milligrams per milliliter and about 50 milligrams per milliliter of said amount of said biocompatible solvent.

4. The injectable composition for fat reduction of claim 2, wherein said amount deoxycholate provides between about 22 milligrams per milliliter and about 42 milligrams per milliliter of said amount of said biocompatible solvent.

5. The injectable composition for fat reduction of claim 2, wherein said amount of phosphtidycholine provides between about 24 milligrams per milliliter and about 28 milligrams per milliliter of said biocompatible solvent.

6. The injectable composition for fat reduction of claim 2, wherein said amount of deoxycholate provides between about 22 milligrams per milliliter and about 26 milligrams per milliliter of said amount of said biocompatible solvent.

7. The injectable composition for fat reduction of claim 2, wherein said amount of phosphtidycholine provides between about 26 milligrams per milliliter and about 27 milligrams per milliliter of said amount of said biocompatible solvent.

8. The injectable composition for fat reduction of claim 2, wherein said amount of deoxycholate provides between about 24 milligrams per milliliter and about 25 milligrams per milliliter of said amount of said biocompatible solvent.

9. The injectable composition for fat reduction of claim 1, further comprising an amount of anesthetic.

10. The injectable composition for fat reduction of claim 9, further comprising an amount of beta adrenergic stimulator.

11. The injectable composition for fat reduction of claim 10, further comprising an amount of alcohol.

12. The injectable composition for fat reduction of claim 9, wherein said amount of anesthetic is selected from the group consisting of: ropivacaine, articaine, benzocaine bupivacaine, chloroprocaine, etidocaine, hexylcaine, lontocaine, lidocatine, levobuivaciaine, mepivacaine, prilocaine, procaine, and tetracaine.

13. The injectable composition for fat reduction of claim 12, wherein said amount of anesthetic comprises an amount of ropivacaine which provides between about 4 cc and about 6 cc in 100 cc of said injectable composition.

14. The injectable composition for fat reduction of claim 10, wherein said amount of beta adrenergic stimulator is selected from the group consisting of: isoproterenol hydrochloride, forskolin, norepinephrine, guarana and clenbuterol.

15. The injectable composition for fat reduction of claim 14, wherein said amount of beta adrenergic stimulator comprises an amount of isuprel which provides between about 4 milligrams and about 6 milligrams in 100 cc of said injectable composition.

16. The injectable composition for fat reduction of claim 11, wherein said amount of alcohol is selected from the group of alcohols consisting of: an amount of benzyl alcohol, an amount of ethanol, an amount of butanol, and an amount of pentantol.

17. The injectable composition for fat reduction of claim 16, wherein said amount of alcohol comprises an amount of benzyl alcohol which provides between about 18 milligrams per milliliter and about 30 milligrams per milliliter of said biocompatible solvent.

18. The injectable composition for fat reduction of claim 2, wherein said amount of biocompatible solvent is selected from the group consisting of: an amount of water, an amount of saline, an amount of water combined with an amount of alcohol, an amount of ethanol, an amount of butanol, an amount of pentanol, an amount of benzyl alcohol, an amount of benzylbenzoate, and an amount of dimethylformamide.

19-42. (canceled)
